(19) United States
(12) Patent Application Publication
(10) Pub. No.: US 2023/0414822 A1
(43) Pub. Date: Dec. 28, 2023

(54) PERSONAL ULTRAVIOLET RESPIRATORY GERM ELIMINATION MACHINE (PUR GEM)

(71) Applicant: Wayne State University, Detroit, MI (US)

(72) Inventor: Nausheen R. Shah, Grosse Pointe Park, MI (US)

(21) Appl. No.: 18/036,789

(22) PCT Filed: Nov. 3, 2021
(86) PCT No.: PCT/US2021/057940

§ 371 Date: May 12, 2023

Related U.S. Application Data
(60) Provisional application No. 63/112,825, filed on Nov. 12, 2020.

(51) Int. Cl. A61L 9/20; A62B 18/00

(52) U.S. Cl. CPC A61L 9/20; A62B 18/003; A61L 2209/12

(57) ABSTRACT

A Germ Eliminating Machine (GEM) having a first spheroid cavity and a second spheroid cavity coupled in series to the first spheroid cavity is set forth. The GEM is configured to pass outside air through the first spheroid cavity and into the second spheroid cavity. The GEM also includes a first UV device configured to provide UV light to the first spheroid cavity, such that UV light repeatedly reflects in the first spheroid cavity, and a second UV device configured to provide UV light to the second spheroid cavity such that UV light repeatedly reflects in the second spheroid cavity. The UV light from the first and second UV devices damage germs in the outside air that passes through the first spheroid cavity and into the second spheroid cavity.

### CROSS-REFERENCE TO RELATED APPLICATIONS

This application claims priority to U.S. Provisional Patent Application No. 63/112,825 filed on Nov. 12, 2020, the contents of which are hereby incorporated in its entirety.

### TECHNICAL FIELD

This disclosure relates generally to germ elimination technology.

### BACKGROUND

The disinfection properties of ultraviolet (UV) radiation in the 200-300 nm have been documented. Many solutions using UV radiation, such as cavity disinfection and whole room decontamination between uses, are in use in various industries (e.g., healthcare).

The elimination of an organism via UV radiation is generally dose-dependent. There have been investigations into the optimal dose required, showing a generally large range of efficacious dosages administered. For example, it has been shown that 222 nm UV radiation is able to inactivate various coronaviruses to the 3-log level with a dose of approximately less than or equal to 2 mJ/cm². It has also been shown that the H1N1 virus requires a dose of 4 mJ/cm² to give a 4-log (99.99%) reduction using 222 nm radiation. Further, it has been shown that irradiation by 254 nm UV provided similar elimination of H1N1. For the Murine Hepatitis Virus coronavirus, a similar dose requirement was found when irradiated with 254 nm UV. 254 nm and 222 nm radiation generally gives similar results for bacterial targets, even though the precise cause of elimination is considered different.

While the elimination of organisms via UV radiation is known, it can be difficult to create UV devices that eliminate or reduce organisms in an economical and efficient manner That is, it can be difficult to economically produce a device that provides the amount of UV energy per unit of area needed to achieve the desired pathogen reductions. For example, in order to produce the desired UV energy per unit of area, devices often employ multiple UV lights. As the amount of UV lights in such devices increase, the costs of such devices can also increase. In turn, the maintenance costs of such devices can also increase if, and how often, the UV lights fail during the device life-span.

Accordingly, there is a need for efficient and economical UV disinfectant devices or machines.

### BRIEF DESCRIPTION OF THE DRAWINGS

FIG. 1 is a planar cross-sectional view of an exemplary germ eliminating device;

FIG. 2 is an illustration of a user employing an exemplary germ eliminating device;

FIG. 3 is a planar cross-sectional view of an exemplary germ eliminating device coupled to a ventilator;

FIG. 4 is a planar cross-sectional view of an exemplary germ eliminating device having K cavities;

FIG. 5 is a graph representing virus elimination of five exemplary germ eliminating devices; and

FIG. 6 is a flowchart illustrating a technique for manufacturing a germ eliminating device.

### DETAILED DESCRIPTION

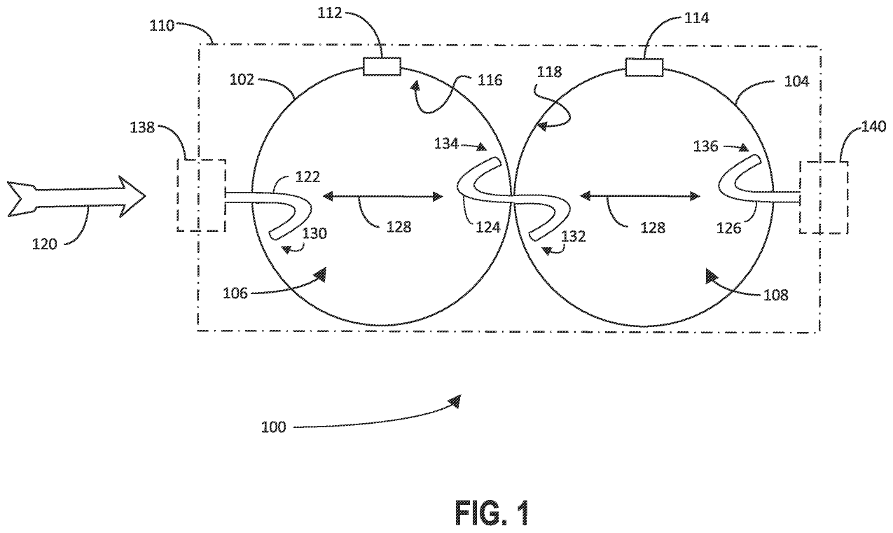

Devices or machines that can safely, efficiently, economically, and continuously disinfect inhaled and/or exhaled air using ultraviolet (UV) radiation with possible pathogen elimination up to 99.99% or more are set forth. As will be discussed in further detail below, such devices or machines may, for example, be configured as personal (e.g., wearable) units or units to “clean” enclosure air (e.g., building rooms, underground bunkers, transportation vehicles, and etc.). The utilized UV radiation may be in the 260 nm range where no ozone is produced, thus minimizing manufacturing costs since mechanical and/or chemical ozone mitigation measures can be avoided. These devices may be portable, wearable, or scaled up to larger sizes as needed. Further, since a utilized UV chamber (see. e.g., FIG. 1) may be self-contained, UV exposure to a user can be avoided.

The manner in which the chambers or cavities of such devices are designed provide an amplification of irradiated UV power by approximately 10-50 times. That is, the irradiated UV power may be amplified by approximately 10-50 times due to the innovative design of the cavities set forth below.

As will be discussed in detail below, in some examples, such devices may be employed as a Personal Ultraviolet Respiratory Germ Eliminating Machine (PUR GEM) or device, which may be worn by a user or attached to devices such as a ventilator exhaust and/or ventilator intake. In other examples, such devices may serve as free-standing portable (or permanent) air disinfection units.

Referring now to FIG. 1, a planar cross-sectional view of an exemplary Germ Eliminating Machine (GEM) 100 or device is shown. The GEM 100 includes a first chamber 102 and a second chamber 104. The first chamber 102 includes a first spheroid cavity 106 and the second chamber 104 includes a second spheroid cavity 108. While the chambers 102, 104 refer to structure, the cavities 106, 108 refer to the openings or voids in the chambers 102, 104.

The first and second cavities 106, 108 are spheroid, though the chambers 102, 104 need not be spheroid. That is, though not shown, the first and second chambers 102, 104 may be a shape different than spheroid while the cavities 106, 108 therein remain spheroid. Further, these chambers 102, 104 may be permanently coupled to each other or, alternatively, be modular such that they may be separated as needed or that additional chambers with cavities (not shown) may be added.

In yet another example, the GEM 100 may instead include a single chamber 110 (shown in phantom), that includes the first and second spheroid cavities 106, 108. That is, instead of two chambers 102, 104 each including the respective spheroid cavity 106, 108, the single chamber 110 may instead be configured to include the first and second spheroid cavities 106, 108. While FIG. 1 illustrates the single chamber 110 as being a parallelogram shape, the single chamber 110 may be a shape different than that shown.

With reference back to the first and second chambers 102, 104 illustrated in FIG. 1, the first chamber 102 has a first ultraviolet (UV) device 112, having a power P, coupled thereto. The UV device 112 projects or passes UV light into the first spheroid cavity 106. Similarly, the second chamber 104 includes a second UV device 114 coupled thereto that passes UV light into the second spheroid cavity 108. The second UV device 114 may have a power P or some other power. The positions of the UV devices 112, 114 shown in FIG. 1 are merely exemplary. That is, the GEM 100 may be constructed in such a manner that the UV devices 112, 114 are positioned differently than that shown in FIG. 1.

A variety of UV devices may be employed. For example, mercury based cold cathode lamps (CCL) may be employed or UV light emitting diodes (LED) may be employed. While mercury CCLs are currently and generally considered cheaper than UV LEDs, UV LEDs may have advantages due to their generally longer lifespan and their ability to produce UV wavelengths more efficient for pathogen elimination.

With continued reference to FIG. 1, the first spheroid cavity 106 is substantially surrounded by a first UV reflective boundary 116 and a second UV reflective boundary 118 substantially surrounds the second spheroid cavity 108. Due to the UV reflective nature of the boundaries 116, 118 and the spheroid shape of the cavities 106, 108, irradiance is generally uniform throughout each spheroid cavity 106, 108. In other words, due to the UV reflective boundaries 116, 118 and the spheroid-shaped cavities 106, 108, UV light passes into each cavity 106, 108 via the respective UV device 112, 114 and repeatedly reflects off the respective boundary 116, 118 such that irradiance is generally uniform.

The UV reflective boundaries 116, 118 may be comprised of a variety of UV reflective materials. For example, polytetrafluoroethylene (PTFE), which is UV resistant and has a reflectance often greater than 95%, may be employed as the first and second UV reflective boundaries 116, 118. Different UV reflective materials, however, may instead be employed. Further, the materials (116, 118) need not necessarily be different than the material that comprises the chambers 102, 104. In other words, each chamber may be comprised or composed of a UV reflective material. In such an instance, a separate boundary material need not be employed since the chamber boundary itself may be adequately UV reflective.

Air 120 (e.g., outside air or patient air) passes through a first air pipe 122 to enter the first spheroid cavity 106 and a second air pipe 124 allows air from the first spheroid cavity 106 to enter the second spheroid cavity 108. A third air pipe 126 allows air to exit the second spheroid cavity 108. Accordingly, air enters the first spheroid cavity 106 where it is irradiated by the first UV device 112. Due to the reflective nature of the first reflective boundary 116 and the shape of the first spheroid cavity 106, air within the first spheroid cavity 106 is repeatedly irradiated via the first UV device 112 to increase germ elimination as it passes through the first spheroid cavity 106.

The innovative design employing the reflective boundary 116 and the spheroid-shaped cavity 106 serves as an enhancement factor for the UV device 112. In other words, for a given UV device having a power P, the spheroid cavity 106 and reflective boundary 116 can increase the power P of UV device by approximately 10-50 times, thus amplifying its ability to eliminate germs.

Air within the first spheroid cavity 106 eventually passes through the second air pipe 124 into the second spheroid cavity 108 where it is once again repeatedly irradiated by UV light, but from the second UV device 114. As such, air 120 that enters the GEM 100 is repeatedly irradiated first as it passes through the first spheroid cavity 106 and again as it passes through the second spheroid cavity 108, thus maximizing germ elimination before the air passes out the third air pipe 126. Germs may include, for example, viruses, bacteria, and/or other infectious agents.

The air pipes 122-126 may be positioned to optimize diffuse turbulent air flow through the cavities 106, 108, thus increasing the probable amount of time any given packet or unit of air is exposed to UV light (direct and reflected). For example, as illustrated in FIG. 1, the first air pipe 122 and the second air pipe 124 are positioned to maximize a distance 128 therebetween since the first and second air pipes 122-124 are on opposite ends of the first spheroid cavity 106. Accordingly, the probable travel time of air through the first spheroid cavity 106 is increased or maximized. For similar reasons, the distance 128 between the second air pipe 124 and the third air pipe 126 is also maximized.

In addition to the positions of the air pipes 122-126, the configurations of the air pipes 122-126 may also be manipulated to optimize diffuse turbulent air flow through the cavities 106, 108. For example, to further optimize diffuse turbulent air flow, each pipe 122-126 may be twisted or curved to minimize the probability of straight-line paths between consecutive air pipes (e.g., 122, 124, or 124, 126) and/or to increase the probable turbulent nature of airflow through each spheroid cavity 106, 108. That is, a bent outlet 130 of the first air pipe 122 may direct air to the first cavity UV reflective boundary material (or chamber wall) 116 to increase the turbulent nature of airflow through the cavity 106. Similarly, a bent outlet 132 of the second air pipe 124 may direct air to the second UV reflective boundary 118 (or chamber wall) to increase the turbulence in the second cavity 108. The twisted nature of the pipes 122-126 increases the diffuse and turbulent nature of air flow through each cavity 106, 108. Accordingly, the chances that a given unit of air will take a straight-line path through each cavity 106, 108 is minimized. In turn, the probable amount of time a given unit of air spends in each cavity is maximized or increased, thus increasing the probable amount of time the given unit of air, and germs contained therein, is bombarded by the direct and reflected UV light.

Inlets of the air pipes 122-126 may also be configured to maximize the turbulent nature of air flow through the cavities 106, 108 as well as to minimize straight-line air flow through the cavities 106, 108. For example, a bent inlet 134 of the second air pipe 124 may be configured to draw air from a portion of the UV reflective boundary 116 adjacent to the second air pipe 124. Similarly, a bent inlet 136 of the third air pipe 126 may be configured to draw air from a portion of the UV reflective boundary 118 adjacent to the third air pipe 126.

While the air pipes 122-126 illustrated in FIG. 1 are curved or bent for reasons discussed above, in some instance they may be a different shape or even a straight-line shape.

With continued reference to FIG. 1, the GEM 100 may include a first air moving device (e.g., a fan or impeller) 138 to increase, control, or otherwise aid the flow of air through the device 100. Additionally, or alternatively, a second air moving device 140 may be employed to aid the movement of air through the device 100. The placement of the first and/or second air moving devices 138, 140, or whether or not they are employed, may depend on the application.

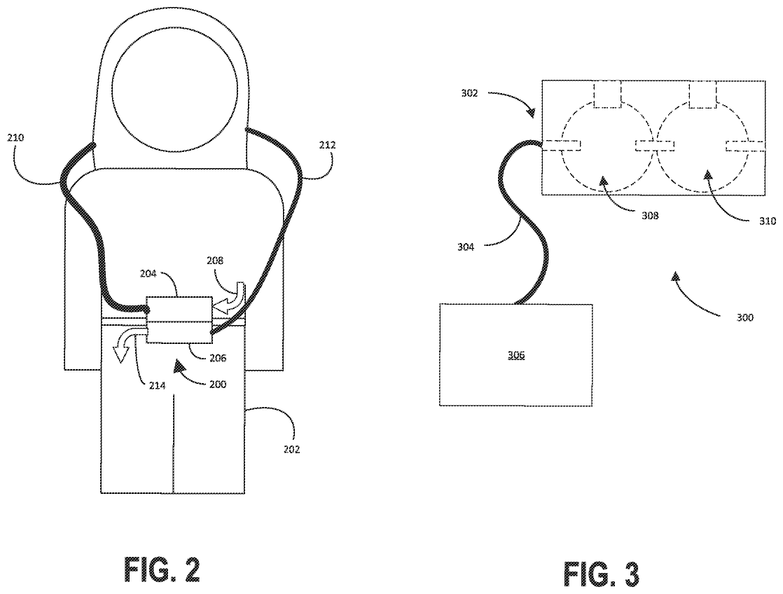

Referring now to FIG. 2, exemplary GEM technology 200 employed by a user 202 is shown. The GEM technology 200 includes a first GEM 204 and a second GEM 206. Each GEM 204, 206 is configured in a manner similar to the GEM 100 of FIG. 1. Though not shown, like the GEM 100 of FIG. 1, the first and second GEMS 204, 206 each include a plurality of spheroid cavities (e.g., two or more spheroid cavities), where each cavity is substantially surrounded by a UV reflective boundary. Further, each GEM 204, 206 includes a plurality of UV devices to treat air passing therethrough. The quantity of UV devices employed will generally depend on the amount of spheroid cavities in each GEM 204, 206. If a GEM employs two cavities (e.g., FIG. 1), a sperate UV device will generally be associated with each cavity. Similarly, if for example five spheroid cavities are employed, the GEM may include five UV devices. It is noted that other devices not shown, such as air moving devices, may also be employed.

With reference back to FIG. 2, the first GEM 204 is configured to clean intake air 208. As cleaned or disinfected air leaves the first GEM 204 it is conveyed through an air supply route 210 and conveyed to the user (e.g., a healthcare worker) 202 such that the user is able to breathe “cleaned” air. The second GEM 206 is employed to “clean” the exhaled air from the user 202. That is, air exhaled from the user 202 is conveyed through an exhaust tube 212 to the second GEM 206, where it is cleaned or disinfected, before it passes out the second GEM 206 as exhaust air 214. Due to the efficiency of the GEM (e.g., the first or second GEM 204, 206), it may serve as personal protection equipment (PPE) that may be worn to eliminate or render innocuous infectious agent(s) in the air.

While FIG. 2 illustrate two GEMs 204, 206, one 204 to treat air inhaled air via the air supply route 210 and one 206 to treat exhaled air through the exhaust tube route 212, the two machines 204, 206 could be manufactured as a single device or machine. That is, a single GEM may be configured to treat both inhaled and exhaled air. Alternatively, a PPE GEM may be configured to treat only inhaled air or exhaled air.

With reference now to FIG. 3, a planar cross-sectional view of an exemplary GEM 300 is shown in the context of another healthcare example. In this example, an intake 302 of the GEM 300 is coupled to an outflow 304 or exhaust of a ventilator 306 to eliminate or render innocuous infectious agent(s) from a patient (not shown). Alternatively, or in addition, the GEM could be coupled to an intake of a ventilator to clean or disinfect patient breathing air. While the GEM 300 employs two air cavities 308, 310, other examples may have greater than two cavities to further maximize germ elimination.

The GEM 300 may be multi-use. That is, the GEM 300 may be employed as a PUR GEM (see, e.g., GEM technology 200 of FIG. 2), but when needed, may be couplable to the ventilator (e.g., GEM 300 of FIG. 3). A multi-purpose GEM may provide cost savings to institutions such as hospitals and also may help with workflows in the same institutions. For example, GEM stock could be stored and used for either patient ventilator needs or for PPE needs.

While healthcare industry examples are set forth herein, the GEM (e.g., GEM 100, 300 or GEM 200) technology may be employed in other industries where “air cleaning” is desired. For example, a GEM may be employed to clean or disinfect a wide variety of enclosure air. With regard to the transportation industry, a GEM may, for example, be employed to clean cabin or interior air in aircraft, trains (e.g., a train car), buses, submarines, and/or automobiles. Further, a GEM may be employed to clean other enclosure air such as air in a building. For example, the GEM may be employed to clean or disinfect air in hospital rooms, hotel rooms, and one or more rooms in a home or underground bunker. In such instances, a GEM may be portably or “permanently” incorporated into the application. Due to the configuration of GEM technology, device size can be minimized so that it may be employed in industries constrained by space requirements.

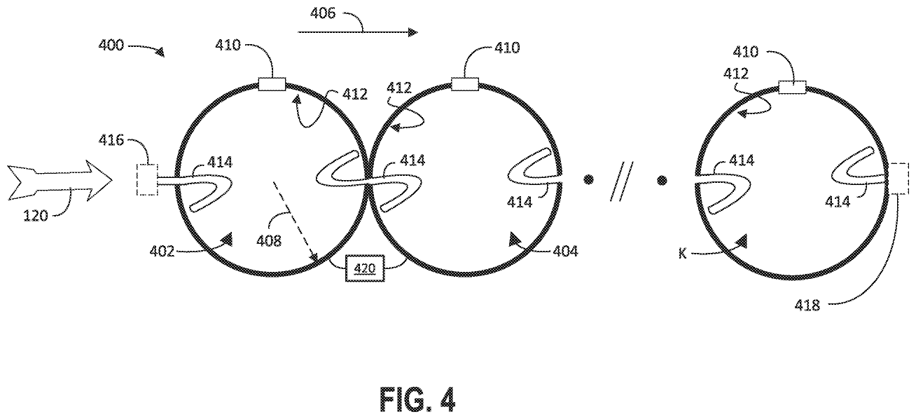

Referring now to FIG. 4, a planar cross-sectional view of another exemplary GEM 400 is illustrated. The GEM 400 includes series of k spherical cavities (402, 404-K) in a disinfection direction 406 (e.g., in an inhalation direction or an exhalation direction). Each spheroid cavity 402, 404-K has a radius r 408, leading to total linear dimension of approximately 2rk=2R. A UV source 410 (e.g., a UV LED) irradiates each respective spheroid cavity (402-K) uniformly due to high UV reflective interior walls 412. Air flow at a specified rate may be propelled via one or more exemplary fans or the like 416, 418. The air is conveyed through the series of spheroid cavities 402-K via a plurality of curved pipes/buffered pipes or ports 414 to prevent, or at least minimize straight-line flow therethrough and to increase the turbulent nature of the flowing air.

One or more of the fans or the like, fan 416 and/or fan 418, may include a tight-weave cloth filter (not shown) covering an opening to and/or from the chamber to serve primarily as a particulate filter. More sophisticated particulate filters may be used if desired. A sensor 420 could be incorporated into circuit design to alert a user if air flow falls below a predetermined level. Further, the same or different sensor could be employed to alert a user or technician of low battery failures, LED device defects, and/or other mechanical or electrical issues that may arise.

Due to the reflectance and shape of the walls 412, a geometric enhancement factor for the UV power in each sphere is produced. This enhancement factor is reduced by defects in the reflecting surface, such as those due to the surface area occupied by the LED 410 and the air vents 414. Therefore, the effective reflectance R_{eff} of the cavity (e.g., cavities 402-K) is given by:

$${R}_{eff} = R ( \frac{S {A}_{s} - {A}_{d e f}}{S {A}_{s}} ) , \qquad ( 2.1 )$$

where R is the reflectance of the walls, SAₛ=4πr² is the surface area of a sphere of radius r, and A_{def} is the total area of defects. Practically, it may be desired to limit A_{def} to be less than approximately 5% of SAₛ. The enhancement due to effectively infinite reflections of light inside the spheroid cavity gives a multiplicative factor to P_{UV}:

$$ε = \frac{1}{1 - {R}_{eff}} \qquad ( 2.2 )$$

A quantity Do may then be computed as follows:

$${D}_{o} = \frac{ε {P}_{U V}}{S {A}_{s}} \frac{{V}_{s}}{{V}_{A F}} = \frac{{P}_{U V}}{3 {V}_{A F ( 1 - {R}_{eff} )}} r , \qquad ( 2.3 )$$

where P_{UV} is the UV source radiation in mW, Vₛ=(4/3)πr³ is the volume of a sphere, and V_{AF} is the required air flow rate in cm³/s. This quantity Do is what would be naively computed as the dose received by air flowing through a cavity (e.g., spheroid cavities 402-K) in units of mJ/cm².

The spherical shape for the UV disinfection cavity 402-K provides excellent spatial integration. Since there are minimal radiation hot or cold spots, the UV dose received by an in-passing air volume becomes effectively independent of path taken through the sphere. However, travel time distributions, not average time, should be taken into account to accurately predict actual disinfection, and in general will reduce the actual pathogen elimination significantly independent of geometry. To compensate for this effect, a series of spheres (402-K) that effectively integrate the air paths temporally is employed, allowing for the achievement of much higher levels of actual disinfection. That is, as shown, while a single spheroid cavity may not effectively integrate air paths temporally, multiple spheroid cavities allow for the effective integration of germ paths temporally.

The time air spends in the spherical cavities 402-K will likely follow an exponential distribution due to its turbulent motion, as has been verified both numerically and experimentally. The probability that a differential volume element of air will spend a time (t) as it flows through a sphere (cavity 402-K) is given by:

$$𝒫 ( t ) = \frac{1}{𝒯} {e}^{- t / 𝒯} , \qquad ( 2.4 )$$

where [symbol] =Vₛ/V_{AF}. The expectation value for pathogen elimination in this volume of air is computed by weighting it by this distribution. A convention of defining pathogen elimination in base 10 means one should convert:

$$𝕊 = {10}^{- D / {D}_{90}} = {e}^{- \frac{D}{{D}_{90}} ln 10} , \qquad ( 2.5 )$$

where D₉₀ is a dose required for 1−log (90%) pathogen elimination in mJ/cm², D is dose received also in mJ/cm², and [symbol] is surviving fraction of pathogen. However, D should be computed using the time that the pathogen actually spends being irradiated. Therefore, the expectation value of a fraction of pathogen particles surviving passage through a sphere is given by

$${𝕊}_{0} = {\int }_{0}^{∞} {e}^{- \frac{ε {P}_{U V} ln 10}{S {A}_{s} {D}_{90}} t} 𝒫 ( t ) dt = \frac{1}{1 + \frac{{D}_{0}}{{D}_{90}} ln 10} . \qquad ( 2.6 )$$

Given the series of k identical spheres, each of radius r, irradiated by P_{UV}, pathogen elimination will be given by

[symbol]

ₖ=1− [symbol] ₀^{k}. (2.7)

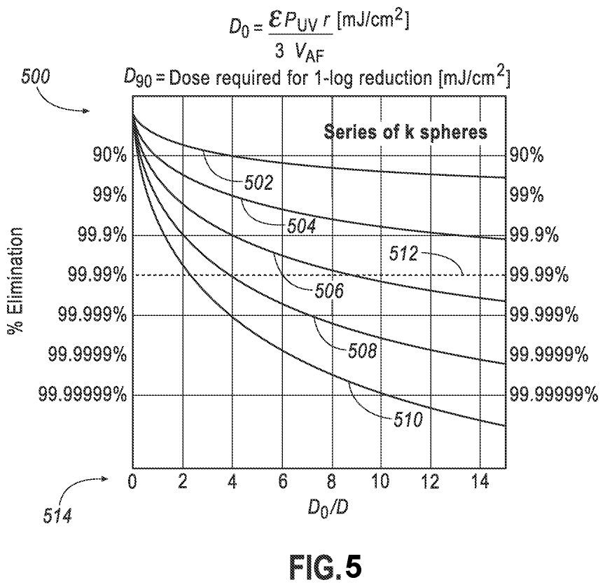

With reference now to FIG. 5, a graph 500 is shown representing possible pathogen elimination as a function of the dimensionless quantity D₀/D₉₀ for multiple configurations of GEMs. The number of spheres (cavities) in each configuration are represented by k. A first line 502 represents a GEM with a k=1 configuration, a second line 504 represents a GEM with a k=2 configuration in series (see, e.g., FIG. 1), a third line 506 represents a GEM with a k=3 configuration in series, a fourth line 508 represents a GEM with a k=4 configuration in series, and a fifth line 510 represents a GEM with a k=5 configuration in series. A log reduction line 512 represents 4-log reduction or generally 99.99% pathogen elimination. Increasing values of D₀/D₉₀ correspond to increase in enhancement factor, P_{UV} or r, or a decrease in air flow rate V_{AF}. As can be seen, initially increasing D₀/D₉₀ leads to significant increase in elimination (see, e.g., initial steep slopes of lines 502-510). However, at a certain point for any number of spheres, gain becomes progressively smaller. For the benchmark elimination of 4-log reduction, it is shown that 3 or more spheres in series are generally needed. It is noted that D₉₀ for SARS-COV2 should be approximately 1 mJ/cm². In this context, x-axis 514 may be read in units of mJ/cm².

An exemplary personal germ eliminating machine (PUR GEM) or device (e.g., FIG. 2) having two spheres in each disinfection direction was created (e.g., GEM 204 and GEM 206 of FIG. 2). That is, the PUR GEM included two spheres in series to treat inhaled air and two spheres in series to treat exhaled air. The radius of each sphere was approximately cm. For the exemplary PUR GEM, an enhancement factor of approximately 18 was validated experimentally. Since PTFE was manually applied to the interior of the spheres (i.e., cavity boundaries), there was likely some degradation in the enhancement factor. Commercial manufacturing could be expected to reduce defects.

Nonetheless, 40 mW UV LEDs were used with an airflow of approximately 30 L/min. This leads to the following SARS-COV2 elimination:

$${𝔼}_{2} = 1 - {𝕊}_{0}^{2} = 1 - {[ \frac{1}{1 + \frac{18 \times 40 mW \times 5 cm}{3 \times 50 \; 0 {cm}^{3} / s \times 1 \frac{mJ}{{cm}^{2}}} ln 10} ]}^{2} = 1 - {[ \frac{1}{1 + 2.4 ln 10} ]}^{2} = 97.65 \% . \qquad ( 2.8 )$$

Significantly smaller spheres may be manufactured commercially. For example, spheres with a radius of r=2 cm (i.e., similar to ping-pong ball size) may be manufactured. Using the same air flow rate as the exemplary PUR GEM, but higher power LEDs, a series of two spheres with r=2 cm would give similar disinfection as the example set forth above, thus illustrating the efficient nature of GEM technology. That is, a 2-sphere GEM, where each sphere has a radius of r/x cm and LEDS having a power of P can achieve similar disinfection as a 2-sphere GEM, where each sphere has a radius r=5 cm and LEDS of power P/x. For example,

$${𝔼}_{2} = 1 - {𝕊}_{0}^{2} = 1 - {[ \frac{1}{1 + \frac{20 \times 80 mW \times 2 cm}{3 \times 500 {cm}^{3} / s \times 1 \frac{mJ}{c {m}^{2}}} ln 10} ]}^{2} = 1 - {[ \frac{1}{1 + 2.13 ln 10} ]}^{2} = 97.14 \% \qquad ( 2.9 )$$

For higher disinfection, five spheres having LEDs of the same power and radius as in equation (2.9) would provide the following pathogen (e.g., SARS-COV2) elimination:

$${𝔼}_{5} = 1 - {𝕊}_{0}^{5} = 1 - {[ \frac{1}{1 + \frac{20 \times 80 mW \times 2 cm}{3 \times 500 {cm}^{3} / s \times 1 \frac{mJ}{{cm}^{2}}} ln 10} ]}^{5} = 1 - {[ \frac{1}{1 + 2.13 ln 10} ]}^{5} = 99.99 \% . \qquad ( 2.1 )$$

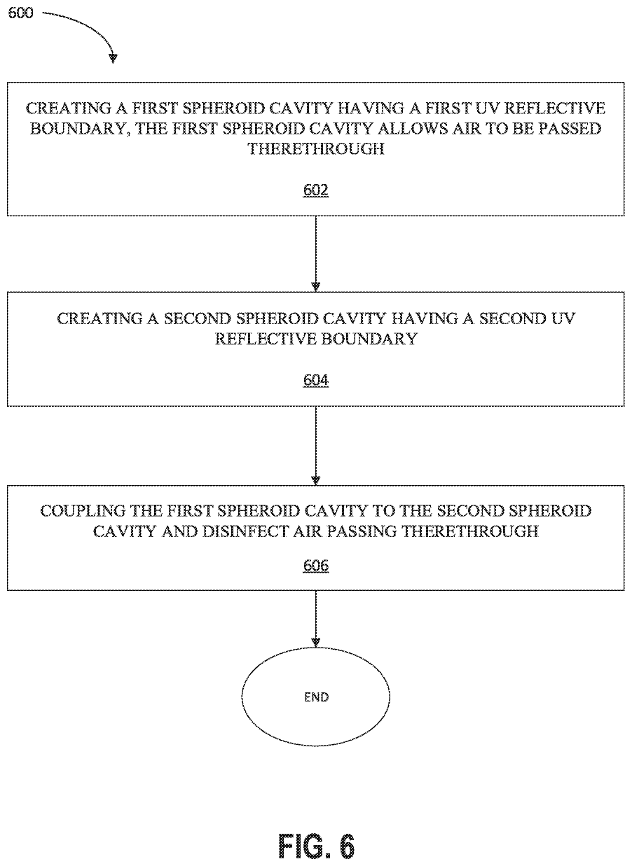

Referring now to FIG. 6, a technique 600 for manufacturing a GEM is set forth. The technique 600 may begin at block 602 with creating a first spheroid cavity having a first UV reflective boundary, the first spheroid cavity allows air to be passed therethrough. Process control may then proceed to block 604 where creating a second spheroid cavity having a second UV reflective boundary is set forth. Once the first and second spheroid cavities are created, process control may continue to block 606 for coupling the first spheroid cavity to the second spheroid cavity such that air passing into the first spheroid cavity passes through the first spheroid cavity and then through the second spheroid cavity, where the GEM is configured to disinfect air passing therethrough. Technique 600 may then proceed to an END.

While not shown, however, the technique may also include: (i) providing a first location where a first UV device passes UV light into the first spheroid cavity and (ii) providing a second location where a second UV device passes UV light into the second spheroid cavity, where the UV light reflecting in the first spheroid cavity and UV light reflecting in the second spheroid cavity damages germs in the air passing therethrough. The UV device locations may be provided prior to coupling the spheroid cavities together.

Still further, the technique may include: (i) providing an air outlet in the first spheroid cavity to direct incoming air to a boundary of the first spheroid cavity adjacent to the air outlet in the first spheroid cavity and (ii) providing an air outlet in the second spheroid cavity to direct incoming air to a boundary of the second spheroid cavity adjacent to the air outlet in the second spheroid cavity. An air intake in the first spheroid cavity may draw air from a boundary of the first spheroid cavity adjacent to the air intake in the first spheroid cavity, while an air intake in the second spheroid cavity may draw air from a boundary of the second spheroid cavity adjacent to the air intake in the second spheroid cavity. The air passing through the GEM passes out the air outlet of the first spheroid cavity through the first spheroid cavity to the air intake in the first spheroid cavity and then out the air outlet in the second spheroid cavity and through the second spheroid cavity to the air intake in the second spheroid cavity before passing out the second spheroid cavity.

The technique may also include creating additional spheroid cavities in series such as creating a third spheroid cavity having a third UV reflective boundary and creating a fourth spheroid cavity having a fourth UV reflective boundary. By coupling the third spheroid cavity to the fourth spheroid cavity, air passing into the third spheroid cavity passes through the third spheroid cavity and then through the fourth spheroid cavity.

With regard to FIGS. 1-6 and the processes, systems, methods, techniques, heuristics, etc. described herein, it should be understood that, although the steps of such processes, etc. have been described as occurring according to a certain ordered sequence, such processes could be practiced with the described steps performed in an order other than the order described herein. It further should be understood that certain steps could be performed simultaneously, that other steps could be added, or that certain steps described herein could be omitted. In other words, the descriptions of processes herein are provided for the purpose of illustrating certain embodiments, and should in no way be construed so as to limit the claims.

Accordingly, it is to be understood that the above description is intended to be illustrative and not restrictive. Many embodiments and applications other than the examples provided would be apparent upon reading the above description. The scope should be determined, not with reference to the above description or Abstract below, but should instead be determined with reference to the appended claims, along with the full scope of equivalents to which such claims are entitled. It is anticipated and intended that future developments will occur in the technologies discussed herein, and that the disclosed systems and methods will be incorporated into such future embodiments. In sum, it should be understood that the application is capable of modification and variation.

When introducing elements of various embodiments of the disclosed materials, the articles “a,” “an,” “the,” and “said” are intended to mean that there are one or more of the elements. The terms “comprising,” “including,” and “having” are intended to be inclusive and mean that there may be additional elements other than the listed elements. Furthermore, any numerical examples in the following discussion are intended to be non-limiting, and thus additional numerical values, ranges, and percentages are within the scope of the disclosed embodiments.

What is claimed is:

1. A Germ Eliminating Machine (GEM) comprising:
a first spheroid cavity having a first ultraviolet (UV) reflective boundary substantially surrounding the first spheroid cavity;
a first UV device configured to project UV light into the first spheroid cavity;
a first air pipe configured to convey air into the first spheroid cavity, wherein the first UV device is configured to damage germs in air passing through the first spheroid cavity;
a second spheroid cavity having a second UV reflective spheroid boundary substantially surrounding the second spheroid cavity;
a second UV device configured to project UV light into the second spheroid cavity; and
a second air pipe configured to convey air from the first spheroid cavity to the second spheroid cavity, wherein the second UV device is configured to damage germs in air passing through the second spheroid cavity.

2. The GEM of claim 1, the first air pipe having a first air pipe outlet to provide air to the first spheroid cavity and the second air pipe having a second air pipe inlet to receive air from the first spheroid cavity, wherein the first air pipe outlet and the second air pipe inlet are configured to create diffuse turbulent air flow through the first spheroid cavity and minimize straight-line airflow through the first spheroid cavity.

3. The GEM of claim 2 further comprising a third air pipe configured to convey air out of the second spheroid cavity, wherein the GEM is configured to (i) cause UV light from the first UV device to repeatedly reflect in the first spheroid cavity and (ii) cause UV light from the second UV device to repeatedly reflect in the second spheroid cavity.

4. The GEM of claim 3 wherein the GEM is configured to be worn by a user and to convey UV disinfected air to the user.

5. The GEM of claim 3 wherein the GEM is configured to be coupled to a ventilator exhaust to disinfect air passing out the ventilator exhaust.

6. The GEM of claim 3 further comprising at least one additional spheroid cavity in series with the first and second spheroid cavities.

7. The GEM of claim 3 further comprising an air moving device to at least aid in conveying air through the first and second spheroid cavities.

8. The GEM of claim 3 further comprising a second air pipe outlet to provide air from the first spheroid cavity into the second spheroid cavity, wherein the first air pipe outlet directs air to a portion of the first UV reflective boundary that is adjacent to the first air pipe outlet, and wherein the second air pipe outlet directs air to a portion of second UV reflective boundary that is adjacent to the second air pipe outlet.

9. The GEM of claim 8 further comprising an air inlet of the second spheroid cavity, wherein the air inlet of the second spheroid cavity is configured to draw air from a portion of the first UV reflective boundary adjacent to the air inlet of the second spheroid cavity, and wherein the second air pipe inlet is configured to draw air from a portion of the first UV reflective boundary adjacent to the second air pipe inlet.

10. A Germ Eliminating Machine (GEM) comprising:
a first spheroid cavity;
a second spheroid cavity coupled in series to the first spheroid cavity, wherein the GEM is configured to pass outside air through the first spheroid cavity and into the second spheroid cavity;
a first ultraviolet (UV) device configured to provide UV light to the first spheroid cavity such that UV light repeatedly reflects in the first spheroid cavity; and
a second UV device configured to provide UV light to the second spheroid cavity such that UV light repeatedly reflects in the second spheroid cavity, wherein UV light from the first and second UV devices damage germs in the outside air that passes through the first spheroid cavity and into the second spheroid cavity.

11. The GEM of 10 further comprising:
a first air pipe having a first air pipe outlet to provide outside air to the first spheroid cavity; and
a second air pipe having a second air pipe inlet to receive air from the first spheroid cavity that is conveyed to the second spheroid cavity, wherein the first air pipe outlet and the second air pipe inlet are configured to create diffuse turbulent air flow through the first spheroid cavity and minimize straight-line airflow through the first spheroid cavity.

12. The GEM of claim 11 wherein the GEM is configured to (i) directly provide disinfected air to a user via a wearable device and (ii) be couplable to at least one of an exhaust of a ventilator and an intake of a ventilator.

13. The GEM of claim 11 wherein the GEM is configured to convey disinfected air to at least one of a building enclosure and an interior of a transportation vehicle.

14. The GEM of claim 11 further comprising:
a first UV reflective boundary surrounding the first spheroid cavity; and
a second UV reflective boundary surrounding the second spheroid cavity.

15. The GEM of claim 14 further comprising at least one additional spheroid cavity coupled in series to the second spheroid cavity.

16. A method of manufacturing a Germ Eliminating Machine (GEM) comprising:
creating a first spheroid cavity having a first ultraviolet (UV) reflective boundary, the first spheroid cavity allows air to be passed therethrough;
creating a second spheroid cavity having a second UV reflective boundary; and
coupling the first spheroid cavity to the second spheroid cavity such that air passing into the first spheroid cavity passes through the first spheroid cavity and then through the second spheroid cavity, wherein the GEM is configured to disinfect air passing therethrough.

17. The method of claim 16 further comprising:
providing a first location where a first UV device passes UV light into the first spheroid cavity; and
providing a second location where a second UV device passes UV light into the second spheroid cavity, wherein the UV light reflecting in the first spheroid cavity and UV light reflecting in the second spheroid cavity damages germs in the air passing therethrough.

18. The method of claim 17 further comprising:
providing an air outlet in the first spheroid cavity to direct incoming air to a portion of the first UV reflective boundary adjacent to the air outlet in the first spheroid cavity, wherein the air outlet of the first spheroid cavity creates diffuse turbulent air flow through the first spheroid cavity and minimizes straight-line airflow through the first spheroid cavity; and
providing an air outlet in the second spheroid cavity to direct incoming air to a portion of the second UV reflective boundary adjacent to the air outlet in the second spheroid cavity, wherein the air outlet of the second spheroid cavity creates diffuse turbulent air flow through the second spheroid cavity and minimizes straight-line airflow through the second spheroid cavity.

19. The method of claim 18 further comprising:
providing an air intake in the first spheroid cavity to draw air from a portion of the first UV reflective boundary adjacent to the air intake in the first spheroid cavity; and
providing an air intake in the second spheroid cavity to draw air from a portion of the second UV reflective boundary adjacent to the air intake in the second spheroid cavity, wherein air passing through the GEM passes out the air outlet of the first spheroid cavity through the first spheroid cavity to the air intake in the first spheroid cavity and then out the air outlet in the second spheroid cavity and through the second spheroid cavity to the air intake in the second spheroid cavity before passing out the second spheroid cavity.

20. The method of claim 19 further comprising:
creating a third spheroid cavity having a third UV reflective boundary, the third spheroid cavity allows air to be passed therethrough;
creating a fourth spheroid cavity having a fourth UV reflective boundary; and
coupling the third spheroid cavity to the fourth spheroid cavity such that air passing into the third spheroid cavity passes through the third spheroid cavity and then through the fourth spheroid cavity, wherein the GEM disinfects air inhaled from the first and second spheroid cavities and disinfects air exhaled to the third and fourth cavities.
